(19) United States
(12) Patent Application Publication
(10) Pub. No.: US 2011/0038830 A1
(43) Pub. Date: Feb. 17, 2011

(54) COSMETIC USE OF PLAKOGLOBIN-TYPE PROTEINS

(75) Inventors: Dominique Bernard, Paris (FR); Lucie Simonetti, Vincennes (FR); Isabelle Castel, Nice (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/809,385

(22) PCT Filed: Dec. 19, 2008
(86) PCT No.: PCT/IB2008/055467

§ 371 Date: Nov. 1, 2010

Related U.S. Application Data
(60) Provisional application No. 61/020,581, filed on Jan. 11, 2008.

(30) Foreign Application Priority Data
Dec. 19, 2007 (FR) 0759996

(51) Int. Cl. A61K 8/64; A61K 38/17; A61K 38/19; A61K 38/10; A61K 38/45; C12Q 1/68; G01N 33/566; G01N 33/68; A61Q 19/08; A61Q 19/00; A61P 17/00

(52) U.S. Cl. 424/85.1; 514/18.8; 424/94.5; 435/6; 435/7.21; 436/86

(57) ABSTRACT

The present invention relates to the use, in particular cosmetic and/or therapeutic use, of plakoglobin, of polypeptides derived from this protein or of analogs thereof, of a nucleic sequence encoding such a polypeptide or of an agent for modulating the activity, the stability or the expression of such a polypeptide, in particular for stimulating terminal epithelial differentiation. The invention also relates to the use of plakoglobin, of polypeptides derived from this protein or of analogs thereof, or of a nucleic sequence encoding such a polypeptide, as a marker for evaluating a state of an epithelium.

The subject of the present invention is the use, in particular cosmetic and/or therapeutic use, of plakoglobin, of polypeptides derived from this protein, for example derived from the proteolysis thereof, or of analogs thereof, of a nucleic sequence encoding such a polypeptide or of an agent for modulating the activity, the stability or the expression of such a polypeptide, in particular for stimulating terminal epithelial differentiation, and in particular for preventing and/or treating aged, and optionally dry, skins.

The invention also relates to the use of plakoglobin, of polypeptides derived from this protein or of analogs thereof, or of a nucleic sequence encoding such a polypeptide, as a marker for evaluating a state of an epithelium, and in particular of the epidermis.

Epithelia are tissues of which the cells are joined to and interlinked with one another and lie on a basal membrane. They form either an external covering, for example at the surface of the skin, or the epidermis, or an internal covering, at the surface of a mucosa. They can also form glands.

More specifically, these epithelia are structures of which the homeostasis results from the use of a finely regulated set of intracellular and extracellular signals acting at all the stages of cell proliferation, migration and differentiation, and also of the synthesis of the various extracellular matrix components. These signals can in particular result from the action of factors produced by keratinocytes.

The maintaining of the correct physiological functions of an epithelium involves in particular terminal epithelial differentiation and/or proteoglycan synthesis.

As regards more particularly the epidermis, it is an epithelium, conventionally divided up into a basal layer of keratinocytes containing, in particular, skin stem cells and constituting the germinative layer of the epidermis, a “spiny” layer constituted of several layers of polyhedral cells placed on the basal layer, a “granular” layer comprising one to three layers said to be of flattened cells containing distinct cytoplasmic inclusions, keratohyalin granules, and finally, a set of upper layers, called horny layer (or stratum corneum) constituted of keratinocytes at the terminal stage of their differentiation, called corneocytes.

The stratum corneum, the outermost part of the skin which performs the function of a barrier between the organism and the environment, and the hair shaft, the emerging part of the hair follicle which constitutes the head of hair, both represent the result of the keratinocyte differentiation process. Epidermal differentiation follows a process of maturation in which keratinocytes from the basal layer differentiate and migrate so as to result in the formation of corneocytes, which are completely keratinized dead cells. This differentiation is the result of perfectly coordinated phenomena which will result in the thickness of the epidermis being kept constant and thus ensure the homeostasis of the epidermis.

Many skin disorders or pathologies can result from a dysfunction of epidermal homeostasis, and in particular of terminal epithelial differentiation of keratinocytes and/or of proteoglycan synthesis.

Thus, the principle modifications regarding the epidermis are a decrease in keratinocyte differentiation, leading to a deficiency of the protein matrix of the cornefied cell, an increase in metalloproteinases, which are proteases that degrade the extracellular matrix and that participate in skin aging, and also a decrease in the synthesis of the various glycosaminoglycans, that participate in cutaneous dryness.

For example, in the case of aged skin, this dysfunction is generally manifested by the appearance of wrinkles (microrelief and deep wrinkles), a loss of elasticity, a rough feel and dryness. From the histological point of view, a flattening of the dermo-epidermal junction and a decrease in thickness of the dermis and of the epidermis are observed. The collagen and glycosaminoglycan content decreases. The barrier function of the skin is impaired. All these phenomena are increased by chronic exposure to the sun as well as dryness of the skin.

Similarly, the dysfunction may be worsened in women, during the menopause.

It is already known that, during the various stages of keratinocyte differentiation, several families of proteins, each having a specific function, are involved. Among these, proteases play an essential role in desquamation, i.e. in the removal of corneocytes at the surface of the epidermis, and transglutaminases participate in crosslinking the proteins which will form the horny envelope of the skin and of the hair shaft.

The present invention results in particular from the characterization, by the inventors, of the expression of the plakoglobin protein in the stratum corneum of the human epidermis, in particular in an aged human epidermis, and more particularly in a dry and aged human epidermis.

Plakoglobin (or gamma-catenin or desmoplakin-3) is a protein of which the precursor form comprises 745 amino acids (SEQ ID NO 2) and has a molecular weight of approximately 81.6 kDa. Post-translational modifications, such as phosphorylations on serine or tyrosine residues (S182 and S665, or Y20), are capable of modulating, respectively, its stability and its physiological activity.

This protein is notable in that it appears to be the only protein expressed both in the desmosomes and in the intermediate junctions. Plakoglobin appears to play the role of an assembling protein by creating a link between the proteins of the desmosomal plaque, such as desmoplakin, and the transmembrane proteins. Variants of this protein resulting from alternative splicing appear to exist; however not all of them have yet been completely characterized.

A deficiency in plakoglobin expression, in a transgenic mouse model, appears to be associated with an arrhythmogenic right ventricular cardiomyopathy, and also with epidermal disorders such as bullous epidermolytic hyperkeratosis. A mutation in the gene encoding this protein is associated with Naxos disease.

Plakoglobin appears to exert a negative effect on cell proliferation in the hair follicle and promotes the catagenic phase (Charpentier et al., J Cell Biol, 2000, 149: 503-19).

On the other hand, to the inventors' knowledge, plakoglobin had not up until now been identified as being a protein of which the expression is increased in the aged, and in particular dry aged, human stratum corneum.

In fact, against all expectations, plakoglobin is found also to be a potential marker for the physiological state of the skin, in particular in terms of skin aging, and more particularity in terms of skin aging and cutaneous dryness.

Thus, as emerges from the tests represented hereinafter, the inventors have noted, unexpectedly, on the one hand, an expression of this protein in the stratum corneum, and on the other hand, a significant increase in its expression during aging of the epidermis, and in particular in aged and dry epidermis.

Consequently, according to one of its first aspects, a subject of the present invention is a cosmetic or alternatively nontherapeutic use of an effective amount of at least one polypeptide derived from plakoglobin and, in particular, having an amino acid sequence encoded by a nucleic acid sequence represented entirely or partly by a sequence represented by SEQ ID NO 1, an analog thereof or a fragment thereof, of at least one nucleic sequence encoding such a polypeptide or of at least one agent for modulating the activity, the stability or the expression of such a polypeptide, as an agent that is of use for stimulating terminal epithelial differentiation, in particular of epidermal type.

In particular, such an agent may be useful for preventing and/or treating aged skins. Optionally, the aged skins may aged and dry skins.

According to another of its aspects, a subject of the present invention is also the use of an effective amount of at least one polypeptide derived from plakoglobin and, in particular, having an amino acid sequence encoded by a nucleic acid sequence represented entirely or partly by a sequence represented by SEQ ID NO 1, an analog thereof or a fragment thereof, of at least one nucleic sequence encoding such a polypeptide or of at least one agent for modulating the activity, the stability or the expression of such a polypeptide, for the preparation of a composition, in particular a therapeutic composition, for stimulating an epithelial differentiation, and in particular a terminal epidermal differentiation.

In particular, such a composition may be intended for preventing and/or treating aged skins. Optionally, the aged skins may be aged and dry skins.

In particular, the compositions considered according to the invention may be for stimulating keratinocyte terminal differentiation.

For the purpose of the present invention, the expression “effective amount” is intended to denote the minimum amount required for the observation of the expected effect, namely a cosmetic effect or a therapeutic effect, it being understood that the effective amounts required for obtaining a cosmetic effect or a therapeutic effect may, as appropriate, be identical or different.

For the purpose of the invention, the term “cosmetic use” is intended to denote a use intended mainly to provide an esthetic effect and/or an effect of comfort.

For the purpose of the invention, the term “therapeutic composition” is intended to denote a composition intended to provide a prophylactic or curative effect with respect to epithelial, and in particular epidermal, disorders recognized as reflecting a pathological state.

For the purpose of the invention, the term “prophylactic” or “preventive” is intended to mean a decreased risk of occurrence of a phenomenon, for example a pathology.

A composition in accordance with the invention may, in particular, be for preventing and/or treating the signs of skin aging of an epidermis or of the lips or of the scalp, optionally associated with signs of dryness.

The term “signs of skin aging” is intended to mean all the modifications of the external appearance of the skin due to aging, whether it is of chronological origin and/or photoinduced, for instance wrinkles and fine lines, wizened skin, lack of elasticity and/or of tonicity of the skin, thinning of the dermis and/or degradation of the collagen fibers, thereby leading to the appearance of soft and wrinkled skin.

Dry skin essentially manifests itself through a sensation of tautness and/or tension. Said skin is also rough to the touch and appears to be covered with scales. When the skin is slightly dry, these scales are abundant but not very visible to the naked eye. When this condition worsens, there are increasingly fewer of these scales but they are increasingly visible to the naked eye.

An aged and dry skin may display a total or partial combination of the above-described signs

A composition in accordance with the invention may, in particular, be for preventing and/or treating thinning of an epidermis and/or a loss of firmness, of elasticity, of density and/or of tonicity of an epidermis and/or the formation of wrinkles and fine lines.

In particular, a composition in accordance with the invention may be for preventing and/or treating the signs of skin aging of chronological origin, of an epidermis.

According to another embodiment, a composition in accordance with the invention may in particular be for preventing and/or treating cutaneous signs of dryness, in particular for preventing and/or treating dehydration of an epidermis.

According to another aspect, the present invention also relates to the use of at least one polypeptide in accordance with the invention, as a tool for screening for biological or chemical compounds capable of modulating, and in particular of inhibiting, the expression and/or the biological activity of said polypeptide.

In particular, it relates to a method for screening for anti-aging active agents, comprising at least the steps consisting in:

a) bringing at least one cell type capable of expressing a polypeptide in accordance with the invention, i.e. plakoglobin, or a derivative thereof, into contact with at least one chemical or biological test compound, under conditions suitable for manifestation of the expression of said polypeptide, and

b) determining the content of said polypeptide.

In particular, the present invention relates to the use of at least one polypeptide in accordance with the invention as a tool for screening active agents, for preventing and/or treating aged and dry skins.

According to yet another of its aspects, the present invention also relates to the use of at least one polypeptide in accordance with the invention, or of at least one nucleic acid sequence encoding said polypeptide, as a tool for characterizing, in vitro or ex vivo, a state of an epithelium, and in particular of an epidermis.

In particular, a use in accordance with the invention allows for characterizing in vitro or ex vivo an aged and, optionally dry, state of an epithelium.

More specifically, according to another of its aspects, the present invention relates to a noninvasive, in particular cosmetic, method for characterizing the surface state of an epithelium, in particular of an epidermis, comprising at least the qualitative or quantitative characterization of the expression and/or of the biological activity of a polypeptide in accordance with the invention, i.e. plakoglobin, or of a derivative or fragment thereof.

In particular, a method according to the invention allows for characterizing an aged, and optionally dry, state of an epithelium.

According to a variant embodiment, the datum or value obtained may be assessed in comparison to a reference datum or value, obtained for example from at least one epithelium, in particular one epidermis, which is different than that which is the subject of the characterization, and the state of which is known.

According to another of its aspects, the present invention is also directed toward a noninvasive, in particular cosmetic, method for characterizing the effectiveness of a cosmetic or therapeutic treatment aiming to compensate for the signs of skin aging, comprising at least the qualitative or quantitative characterization of the expression and/or of the biological activity of a polypeptide in accordance with the invention, i.e. plakoglobin, or of a derivative or fragment thereof.

In particular, a method according to the invention allows for characterizing the effectiveness of a cosmetic or therapeutic treatment aiming to compensate for the signs of a skin aging and, optionally cutaneous dryness.

According to a variant embodiment, the datum obtained at the end of the characterization may also be examined in comparison to a reference value or datum. This reference value or datum may be a datum obtained from the epithelium, in particular from the epidermis, that is to be subjected to the treatment, prior to the administration of said treatment or within a shorter chronological time in relation to the treatment start date.

As emerges from the description which follows, the methods according to the invention are particularly advantageous since their implementation does not require an invasive procedure.

The methods of the invention may be carried out in vitro, ex vivo or in vivo.

Indeed, the localization, by the inventors, of the new biomarker for aging, in particular for aging and dryness, namely plakoglobin, in the stratum corneum makes a quantitative or qualitative characterization of the expression of this protein possible by mere topical sampling. The sampling method may, for example, be a stripping technique consisting in applying, to the epithelium under consideration, such as an epidermis, a portion of adhesive tape. On detaching this adhesive tape, a fraction of the epithelium, for example an epidermal fraction, is removed. After protein extraction, said fraction is then analyzed by conventional methods, such as immunoenzymatic assay, or more particularly Western-blot analysis.

Polypeptide Definition

According to one embodiment, a polypeptide suitable for the invention may have an amino acid sequence represented entirely or partly by a sequence represented by SEQ ID NO 2, or an analog thereof, or a fragment thereof.

For the purpose of the present invention, the term “plakoglobin” is intended to denote, in general, unless otherwise indicated, the sequence (SEQ ID NO 2) of the protein having or not having undergone post-translational modifications, such as cleavage or phosphorylation on the serine residues at position 182 or 665 or on the tyrosine residue at position 20, which may or may not be capable of modifying its apparent molecular weight or its isoelectric point, and also the variants resulting from alternative splicing.

It is, moreover, known that the primary sequence of a polypeptide, i.e. the succession of the amino acids, determines sites specifically recognized by protease-type enzymes, such as trypsin, which, once the recognition of these sites has become effective, will induce cleavage of the polypeptide by proteolysis. This proteolysis results in the generation of various peptides, or proteolytic fragments, of the plakoglobin.

The inventors have detected the presence of such peptides in the stratum corneum.

Consequently, the invention also extends to the proteolytic fragments of plakoglobin.

Thus, according to one particular embodiment, a polypeptide suitable for the invention may have an amino acid sequence chosen from SEQ ID NO 3, SEQ ID NO 4 and SEQ ID NO 5, and mixtures thereof.

The term “analog of a polypeptide” is intended to denote any polypeptide exhibiting a sequence homology, in particular with respect to one of the characteristic sequences of said polypeptide, and also a biological activity of the same nature.

This analog may be a peptidomimetic agent.

The homology may be at least 85%, for example at least 90%, and for example at least 95%. The homology may be determined by visual comparison or by means of any computer tool generally used in the field, such as the BLAST programs available on www.ncbi.nlm.nih.gov and used with the default parameters.

The sequence homology may result from modifications derived from mutation or variation in the sequences of the peptides according to the invention, originating either from the deletion or from the insertion of one or more amino acids, or from the substitution of one or more amino acids in the characteristic sequences of a polypeptide according to the invention.

For the purpose of the invention, the term “polypeptide fragment” is intended to denote any portion of a polypeptide in accordance with the invention comprising at least 4, at least 6, in particular at least 8, and more particularly at least 12 consecutive amino acids of said polypeptide, and a substantially similar biological activity.

The term “characteristic sequence of the polypeptide” is, from the viewpoint of plakoglobin, intended to be directed in particular toward the sequence represented by SEQ ID NO 2.

In general, the polypeptide analogs may comprise conservative substitutions relative to the amino acid sequence of the natural polypeptide.

Several of these modifications may be combined.

By way of example of mutations that may be considered in the present invention, mention may be made, nonexhaustively, of the replacement of one or more amino acid residues with amino acid residues having a similar hydropathic index, without however substantially affecting the biological properties of the polypeptide.

The hydropathic index is an index assigned to amino acids as a function of their hydrophobicity and of their charge (Kyte et al. (1982), J. Mol. Biol., 157: 105).

A polypeptide or analog that is also covered by the present invention may be a polypeptide having undergone one or more post-translational modification(s).

The term “post-translational modification(s)” is intended to encompass all the modifications that a peptide or a protein is capable of undergoing at the end of its synthesis in a cell, such as, for example, one or more phosphorylation(s), one or more thiolation(s), one or more acetylation(s), one or more glycosylation(s), one or more lipidation(s), such as a farnesylation or a palmitoylation, a structural rearrangement of the type involving the formation of disulfide bridges and/or cleavage within the peptide sequence.

The analog has, moreover, substantially the same biological activity as the natural polypeptide.

According to one embodiment, a polypeptide suitable for the implementation of the invention may also be a natural or synthetic polypeptide, as appropriate, capable of being obtained after enzymatic or chemical lysis of plakoglobin, or by chemical or biological synthesis, or by extraction from a biological tissue, for instance the skin, expressing this polypeptide naturally or after transfection thereof, and also the various post-translational forms of said polypeptide, or else any natural or synthetic polypeptide of which the sequence completely or partially (entirely or partly) comprises an amino acid sequence mentioned above, for example the variants and the analogs.

Those skilled in the art can obtain a polypeptide in accordance with the invention by means of recombinant DNA-based methods, for instance those described in the manual “Molecular Cloning—A Laboratory Manual” (2nd edition), Sambrook et al., 1989, Vol. I-III, Coldspring Harbor Laboratory, Coldspring Harbor Press, NY, (Sambrook).

According to another embodiment, a polypeptide suitable for the implementation of the invention may also be a polypeptide as defined above, in which at least one residue has been replaced with an amino acid residue having a similar hydropathic index, as defined above.

According to another embodiment, a polypeptide suitable for the implementation of the invention may also be a polypeptide as defined above, fused with another polypeptide, a hydrophilic or hydrophobic targeting agent, a bioconversion precursor, or a luminescent, radioactive or colorimetric labeling agent.

In a nonlimiting manner, mention may be made, as an example of compounds that can be coupled with a polypeptide in accordance with the invention, of fluorescent proteins such as Green Fluorescent Protein, fluorescent chemical compounds such as rhodamine, fluorescein or Texas Red®, phosphorescent compounds, radioactive elements, such as ³H, ¹⁴C, ³⁵S, ¹²¹I or ¹²⁵I, or colorimetric labeling agents such as chromogenic substrates sensitive to the action of galactosidase, of peroxydase, of chloramphenicol acetyltransferase, of luciferase or of alkaline phosphatase.

Depending on the nature of the compounds that can be coupled with a polypeptide in accordance with the invention, the coupling may be performed by chemical methods, in particular by means of reactive chemical functions, or by molecular biology methods known to those skilled in the art.

Definition of Nucleic Acid Sequences

According to one embodiment, the present invention also relates to nucleic acid sequences encoding a polypeptide of the invention and to the employment thereof in the various uses and methods in accordance with the invention.

Thus, the present invention also relates to the use of nucleic acid, in particular deoxyribonucleic acid or ribonucleic acid, sequences encoding a polypeptide in accordance with the invention, in particular the sequences corresponding at least to a nucleic acid sequence represented by SEQ ID NO 1, analogs thereof or a fragment thereof, for the preparation of a composition in accordance with the invention.

For the purpose of the present invention, the term “nucleic acid sequence fragment” is intended to denote a nucleic acid sequence encoding all or part of a polypeptide in accordance with the invention, or an analog of said polypeptide, and in particular a nucleic acid sequence represented by SEQ ID NO 1 or an analog thereof.

The expression “analog of a nucleic acid sequence” is intended to denote any nucleic acid sequence possibly resulting from the degeneracy of the nucleic acid code, and encoding a polypeptide with a sequence identical or analogous to that of the polypeptide encoded by said nucleic acid sequence.

The nucleic acid sequences may be derived from all possible origins, i.e. either of animal, in particular mammalian, and even more particularly human, origin, or of plant origin, or of microbial origin (viruses, phages, bacteria, inter alia) or else of fungal origin, without prejudice regarding whether or not they are naturally present in said organism of origin.

In the case in point, the invention also relates to the use of isolated and purified nucleic acid fragments encoding the polypeptides considered according to the invention.

A nucleic acid sequence in accordance with the invention may comprise a sense, antisense or interfering sequence corresponding to a sequence encoding a polypeptide in accordance with the invention.

Thus, the present invention also relates to the use of nucleic acid, in particular deoxyribonucleic acid or ribonucleic acid, sequences encoding a polypeptide in accordance with the invention.

The nucleic acid sequences according to the invention may in particular be used for preparing the corresponding sense or antisense ribonucleic acid sequences.

A subject of the invention is also the use of any polynucleotide, having a ribonucleic or deoxyribonucleic acid sequence, comprising a sense or antisense sequence, in particular small interfering RNA (siRNA), corresponding at least to the nucleic acid sequence SEQ ID NO 1 or an analog thereof.

Modulating Agent

According to another embodiment, the invention relates to the use of an agent for modulating the expression and/or the stability and/or the activity of a polypeptide in accordance with the invention.

For the purpose of the invention, the term “modulate” is intended to mean, in relation to a given effect, the action of stimulating or inhibiting this effect.

For the purpose of the present invention, the expression “modulating agent or chemical or biological compound capable of modulating the biological activity and/or the expression” is intended to mean any compound capable of acting, directly or indirectly, on at least one polypeptide in accordance with the invention, or a nucleic acid sequence encoding the latter, or on an element of an intracellular or extracellular signaling pathway, or of a metabolic pathway, involving said polypeptide, or on an element involved in regulating the transcription and/or the translation of a nucleic acid sequence encoding said polypeptide, and also in regulating the stability thereof.

The term “biological activity” is intended to denote, in particular from the viewpoint of plakoglobin, the biological activity of the protein represented by the sequence SEQ ID NO 2, of the mature form, and also of the protein having undergone or not having undergone phosphorylations resulting, possibly, in apparent molecular weight variants, and also variants resulting from alternative splicing.

This modulating agent may be an agent for activating or inhibiting the gene or protein expression of a polypeptide of the invention, or else an agent for regulating the stability of said polypeptide.

By way of nonlimiting illustration of the agents for activating the gene expression, mention may in particular be made of 5AzadC, trichostatin A, AML1-ETO, PML-RAR (alpha), PLZF-RAR (alpha), dexamethasone, TGF beta or MnSOD.

By way of nonlimiting illustration of the agents for activating the protein expression, mention may in particular be made of EGF and the farnesyl transferase inhibitor (FTI-277).

By way of nonlimiting illustration of the agents for inhibiting the gene expression, mention may in particular be made of HMGN1, HOXD3, Brn-3b, TNF-alpha, TCDD, alpha-3, beta-1 integrin, cadherin 11 or ALK-1.

By way of nonlimiting illustration of the agents for inhibiting the protein expression, mention may in particular be made of tretinoin (ATRA), (+)-catechin (CAT) or Wnt-11.

By way of nonlimiting illustration, among the agents for regulating the stability, mention may in particular be made of compounds for stimulating proteolytic degradation, such as proteases, ion chelators, sulfonic derivatives, urea derivatives, reducing agents, alpha- or beta-hydroxy acids, ascorbic acid or nicotinamide.

In particular, the modulating agent may be an inhibitor of the gene expression of the polypeptides according to the invention.

According to a preferred embodiment, the modulating agent is an agent for reducing the stability of the polypeptides in accordance with the invention by stimulating the proteolytic degradation thereof.

The present invention relates, in addition, to a method for screening for biological or chemical compounds or for physicochemical factors capable of modulating a biological activity of a polypeptide according to the invention, comprising at least the steps consisting in:

a) bringing at least one polypeptide in accordance with the invention into contact with at least one chemical or biological test compound, and/or subjecting said polypeptide to said physicochemical factor, under conditions suitable for the manifestation of said biological activity of said polypeptide, and

b) determining said biological activity of said polypeptide.

In such a method, the biological activity of the polypeptide, in particular its epithelial differentiation activity, and especially its terminal epidermal differentiation activity, especially in relation to the keratinocytes, may be determined by any method known to those skilled in the art.

For example and in a nonlimiting manner, mention may be made of methods of cell culture followed by characterization of differentiation markers, such as, for example, keratin 10 or filaggrin, or of proliferation markers, such as, for example, KI 67 and PCNA.

According to one embodiment, the biological activity of the polypeptide may be compared to a reference value.

A reference value may be obtained by measuring the biological activity of the polypeptide in the absence of any biological or chemical test compound or physicochemical test factor.

In the event that this reference value measurement is carried out prior to the use of the biological or chemical test compound or of the physicochemical test factor, the method according to the invention may in addition make it possible, where appropriate, to assess the potential effectiveness of said compound.

This biological activity may not be affected by the presence of said compound or, on the other hand, may be inhibited or stimulated.

In the event that an inhibitory effect is noted, the compound tested is capable of being used, for example, as an anti-aging active agent.

Such a compound may be in particular used as an anti-aging active agent and as an active agent favoring the cutaneous moisturization.

A method in accordance with the invention may be carried out on an isolated cell sample, obtained either from a skin biopsy or from cells in culture.

Advantageously, by way of a cell sample suitable for the invention, mention may be made of a keratinocyte sample.

Advantageously, a polypeptide used in a method according to the present invention may be plakoglobin.

The present invention also relates to a method for screening for biological or chemical compounds capable of modulating the expression of a polypeptide in accordance with the invention, comprising at least the steps consisting in:

a) bringing at least one cell type capable of expressing a nucleic acid sequence encoding said polypeptide in accordance with the invention into contact with at least one chemical or biological test compound, under conditions suitable for the manifestation of the expression of said sequence, and

b) determining the expression of said nucleic acid sequence.

The expression of a nucleic acid sequence can be determined, for example, by means of oligonucleotide probes, by any protocol known to those skilled in the art.

By way of example of methods for detecting a nucleic acid sequence, mention may be made of the quantitative (Q-PCR) or nonquantitative polymerase chain reaction (PCR), in the presence or absence of reverse transcriptase (RT-PCR or Q-RT-PCR), of Northern blotting, of the ribonuclease protection assay method, of methods with DNA chips, of methods with transcriptome chips, of methods with oligonucleotide chips, and of in situ hybridization methods.

By way of example of agents suitable for the detection of a nucleic acid sequence, and in particular of mRNA, mention may be made of labeled nucleic acid probes that can hybridize to said sequence.

Such a nucleic acid probe can be readily obtained by any method known to those skilled in the art.

Thus, the nucleic acid sequences in accordance with the invention may be used to prepare sense and/or antisense oligonucleotide primers, which hybridize, under high stringency conditions, to the sequence SEQ ID NO 1 or an analog thereof.

The expression of a nucleic acid sequence in accordance with the invention may be compared to a reference value obtained, for example, by carrying out a method in accordance with the invention in the absence of test compound.

The expression of a nucleic acid sequence may also be determined, indirectly, by determining the expression of the polypeptide encoded by said sequence, by means of any technique known in the field, such as Western blotting, ELISA, the Bradford or Lowry method, or as indicated hereinafter.

The present invention also relates to a method for screening for biological or chemical compounds, or even for anti-aging active agents, capable of modulating the expression of a polypeptide in accordance with the invention, comprising at least the steps consisting in:

a) bringing at least one cell type capable of expressing a polypeptide in accordance with the invention into contact with at least one chemical or biological test compound, under conditions suitable for the manifestation of the expression of said polypeptide,

b) determining the content of the polypeptide, and

c) comparing said content determined in step b) to the content of said polypeptide determined in the absence of chemical or biological test compound.

The comparison carried out in step c) may make it possible to deduce information regarding the suitability of said test compound for modulating the expression of a polypeptide in accordance with the invention.

In particular, a method according to the invention may allow for screening active agents for preventing and/or treating aged, and optionally, dry skins.

A method in accordance with the invention may be carried out on an isolated cell sample.

The determination of the content of the polypeptide in accordance with the invention may be carried out by means of any method known to those skilled in the art.

By way of methods for detecting a polypeptide, mention may be made of Western blotting, slot blotting, dot blotting, ELISA (Enzyme Linked Immunosorbent Assay) methods of the singleplex or multiplex type, proteomics or glycomics methods, staining polypeptides in a polyacrylamide gel with a silver-based stain, with Coomassie blue or with SYPRO, immunofluorescence, UV absorption, immunohistochemical methods in conventional, electron or confocal microscopy, FRET (fluorescence resonance energy transfer), TR-FRET (time resolved FRET) methods, FLIM (fluorescence lifetime imaging microscopy) methods, FSPIM (fluorescence spectral imaging microscopy) methods, FRAP (fluorescence recovery after photobleaching) methods, reporter-gene methods, AFM (atomic force microscopy) methods, surface plasmon resonance methods, microcalorimetry methods, flow cytometry methods, biosensor methods, radioimmuno-assay (RIA) methods, isoelectric focusing methods, and enzyme assays, methods using peptide chips, sugar chips, antibody chips, mass spectrometry methods, and SELDI-TOF spectrometry methods (Ciphergen).

The methods in accordance with the invention may be carried out on a sample, for example an isolated sample, of epithelium, in particular of epidermis, obtained from a skin biopsy or from an epithelial cell model, for example an epidermal cell model, or more advantageously from a noninvasive surface removal, in particular with adhesive tape (stripping tape), of stratum corneum or by simple washing.

A sample of epidermis can be taken by any method known to those skilled in the art.

These methods may be carried out by “stripping” techniques.

These strippings are sticky surfaces applied to the surface of the epidermis, such as Blenderm® from 3M, D'squam (commercial adhesive from CuDERM), cyanoacrylate glue or the varnish stripping method. By virtue of these strippings, the adherent corneocytes and the content of their intercellular spaces can be sampled and subsequently subjected to an extraction which makes it possible to access the protein content.

The taking of a sample suitable for the method may also be carried out more directly by “washing” the skin surface by means, for example, of accessories of the vane turbine type, of the spiral cell type (as described in patent FR 2 667 778) combined with a fluid circuit, or simply by addition/removal of a drop of buffer at the surface of the skin.

By way of indication, other sampling methods suitable for implementing the invention may be mentioned, such as methods based on scraping the upper part of the stratum corneum by means of a twin blade system. This technique makes it possible to collect squamae which can then be directly analyzed by various techniques in order to determine the mineral, amino acid or lipid contents.

It is understood that all the cosmetic or therapeutic compositions considered according to the invention use a physiologically acceptable medium.

For the purpose of the present invention, the term “physiologically acceptable medium” is intended to denote a medium suitable for the application of a composition to an epithelium or a keratin material, such as the skin, the scalp, the lips, the mucous membranes and keratin fibers such as the hair, the nails and body hairs, or, where appropriate, by oral or parenteral administration.

For the purpose of the present invention, the term “therapeutic” is intended to denote a composition that can be used in the context of a prophylactic and/or curative treatment, or of a method for evaluating a state of an epithelium, and in particular of the epidermis.

According to another embodiment, a cosmetic or therapeutic composition in accordance with the invention may also comprise at least one cosmetic and/or therapeutic active agent.

As examples of active agents that can be used in the context of the present invention, mention may be made of cosmetic oils, such as silicone oils, plant oils of the triglyceride type, hydrocarbon-based oils such as Parleam oil and esters of fatty acids and of fatty alcohols.

It may also be possible to use other active agents which make it possible to improve the condition of the skin, such as hydrating or moisturizing active agents or active agents which make it possible to improve the natural lipid barrier, such as ceramides, cholesterol sulfates and/or fatty acids, and mixtures thereof.

It may also be possible to use enzymes which have an activity on the skin, such as proteases, lipases, glucosidases, amidases, cerebrosidases and/or melanases, and mixtures thereof.

As other examples of active agents suitable for implementing the present invention, mention may be made of analgesic active agents, anti-yeast active agents, antibacterial active agents, antiparasitic active agents, antifungal active agents, antiviral active agents, steroidal anti-inflammatory active agents, anesthetic active agents, antipruritic active agents, keratolytic active agents, free-radical scavenger active agents, antiseborrhoeic active agents, antidandruff active agents, anti-acne active agents, active agents intended for preventing aging of the skin and/or for improving the condition thereof, anti-dermatitis active agents, antiirritant active agents, immunomodulatory active agents, active agents for the treatment of dry skin, antiperspirant active agents, antipsoriatic active agents, active agents for protecting against UV, antihistamine active agents, cicatrizing active agents, self-tanning active agents, antioxidants such as green tea or active fractions thereof, glycerol, laponite, caffeine, aromatic essential oils, colorants, depigmenting active agents, liporegulators, emollient, refreshing, deodorizing, desensitizing, bleaching or nourishing active agents, active agents for reducing skin differentiation and/or proliferation and/or pigmentation, and mixtures thereof.

In general, any composition of the invention may be applied to the skin (on any skin region of the body) or to the mucous membranes (buccal, jugal, gingival, genital, conjunctival, etc.).

In a known manner, a cosmetic composition may also contain adjuvants which are customary in the cosmetics field, such as hydrophilic or lipophilic gelling agents, hydrophilic or lipophilic additives, preservatives, antioxidants, solvents, fragrances, fillers, screens, odor absorbers and dyestuffs.

The amounts of the various constituents of the compositions according to the invention are those conventionally used in the fields under consideration.

The amount of chemical or biological compound or of polypeptide, nucleic acid sequence or modulating agent in accordance with the invention contained in a composition according to the invention, also referred to as “effective amount”, of course depends on the nature of the compound and on the desired effect, and may therefore vary to a large extent.

To give an order of magnitude, a composition may contain a modulating agent in accordance with the invention or a polypeptide in an amount representing from 0.00001% to 50% of the total weight of the composition, in particular in an amount representing from 0.001% to 10% of the total weight of the composition, and more particularly in an amount representing from 0.1% to 1% of the total weight of the composition.

A composition according to the invention may be more particularly intended for reducing and/or treating conditions that may cause deterioration of the state of an epithelium, and in particular of an epidermis, and in particular the reducing and/or the treatment of skin aging and, optionally, cutaneous dryness.

A state of an epithelium covered by the present invention may be a state linked to a dysfunction of terminal epithelial, in particular epidermal, differentiation, especially of keratinocytes.

Such a state may be of chronological origin (i.e. linked to the time elapsed, such as skin aging) and/or a sign of a skin disorder, linked, for example, to photoaging.

Thus, a composition in accordance with the invention, especially a cosmetic composition, may in particular be for preventing and/or treating thinning of an epidermis and/or a loss of firmness, of elasticity, of density and/or of tonicity of an epidermis and/or the formation of wrinkles and fine lines.

According to another embodiment, a composition in accordance with the invention, in particular a cosmetic composition, may in particular be for preventing and/or treating cutaneous signs of dryness, in particular for preventing and/or treating dehydration of an epidermis.

A composition of the invention may also be for preventing and/or treating disorders of the barrier function of an epidermis.

According to another embodiment, a composition in accordance with the invention, in particular a cosmetic composition, may be for preventing and/or treating signs of epidermal aging.

In particular, such a composition may be intended for preventing and/or treating aged skins, optionally associated with dryness.

A composition in accordance with the present invention, in particular a therapeutic composition, may be more particularly for use in the treatment of a skin disorder such as a skin hydration disorder, for instance xerosis, parakeratosis, hyperkeratosis, ichthyosis, psoriasis, atopic dermatitis, eczema, rosacea, lichen, pruritus, a skin pathology having an inflammatory component or resulting from an impairment of the immune response, desquamation, disruption of melanogenesis or of sebogenesis, alopecia, hirsutism, a cicatrization disorder, or a skin disorder involving secretion and cell invasion process phenomena, in particular in the context of malignant or benign neoplasias.

According to another aspect, the present invention also relates to the use of at least one polypeptide in accordance with the invention or of at least one nucleic acid sequence encoding said polypeptide, as a tool for characterizing, in vivo or ex vivo, a state of an epithelium, and in particular of an epidermis.

In particular, a use according to the invention allows for characterizing in vitro or ex vivo an aged state of an epithelium.

More particularity, a use according to the invention allows for characterizing in vitro or ex vivo of an aged state of an epithelium, optionally associated with dryness.

By way of example, it is possible to characterize, according to the invention, a state of an epithelium chosen from desquamation, ichthyosis, hyperkeratosis, dryness of an epidermis, chronological aging or photoaging.

Thus, as specified above, according to another of its aspects, the present invention relates to noninvasive methods for characterizing the surface state of a nonpathological epidermis or else the effectiveness of a cosmetic or therapeutic treatment directed to qualitatively or quantitatively characterizing the expression of plakoglobin, or of a derivative or fragment thereof.

In particular, a method according to the invention allows for characterizing an aged state of an epidermis, optionally associated with dryness.

These methods are particularly advantageous since their implementation does not require obligatory recourse to a surgical technique for carrying out such a characterization. An extract of the epidermis can thus be obtained by simple stripping and directly analyzed by a conventional analytical technique, in particular as described above.

According to one embodiment, a method for characterizing a state of an epithelium, for example an epidermis, comprises at least the steps consisting in:

a) determining, in a sample of said epithelium, the content of a polypeptide in accordance with the invention, or of a nucleic acid sequence encoding said polypeptide, and

b) comparing said content determined in step a) to a reference value.

Advantageously, such a method allows for characterizing an aged state of an epidermis. Optionally, the method allows for characterizing an aged and dry state of an epidermis.

Advantageously, a method of the invention is noninvasive.

A method of the invention is advantageously carried out on an isolated sample.

According to one embodiment, a method according to the invention may be carried out on a sample of epithelium, and in particular of epidermis, taken from an individual.

A method according to the invention may also be carried out on a sample of epithelium, and in particular of epidermis, taken from an epithelial cell model, in particular an epidermal cell model, or from a reconstructed isolated skin in order to qualify the state thereof.

A sample of epithelium may be taken by any method known to those skilled in the art.

A method according to the invention may be carried out in vivo, in vitro or ex vivo.

A reference value may, for example, be a content of polypeptide or of nucleic acid sequence determined on a sample of epidermis taken from an epithelium, and in particular from normal skin, i.e. skin that is satisfactory from a physiological point of view, like, for example, young skin and, as appropriate, normally hydrated.

A reference value may be measured in parallel with or following the determination of said content of a polypeptide or of a nucleic acid sequence.

A comparison of a determined content with a reference value may make it possible to evaluate a deviation relative to this value.

The analysis of the intensity and/or of the nature of this deviation (negative or positive) may be informative with regard to the state of the epidermis.

The characterization of a state of an epidermis may be indicative of a possible skin disorder which may be corrected by the use of compounds capable of modulating the expression of a polypeptide of the invention.

According to one embodiment, a method according to the invention may be implemented in a method for the in vivo, in vitro or ex vivo diagnosis of a presumed disorder of an epithelium, and in particular of the epidermis, in an individual.

For example, a state of an epithelium to be evaluated may be chosen from desquamation, ichthyosis, hyperkeratosis, dryness of an epidermis, chronological aging and photoaging.

A polypeptide suitable for carrying out a method according to the invention may advantageously be plakoglobin.

The determination of the content of polypeptide in accordance with the invention or of nucleic acids in accordance with the invention in a sample of epidermis may be carried out by any protocol known to those skilled in the art.

By way of methods for detecting a polypeptide, mention may be made of those mentioned above.

Thus, it is possible to envision detecting the presence of a polypeptide in accordance with the invention by means of an antibody, where appropriate in a labeled form.

An antibody that can be used as a tool for evaluating a state of an epidermis can be obtained by any method known to those skilled in the art, as described in “Antibodies: A Laboratory Manual”, Cold Spring Harbor Laboratory Press, Cold Spring Harbor, N.Y. (1990). Advantageously, the antibodies used may be recombinant antibodies such as those developed by the company Antibodies-by-design.

A nucleic acid sequence suitable for implementing a method according to the invention may advantageously be a nucleic acid sequence encoding plakoglobin, for example of mRNA type.

By way of example of methods for detecting nucleic acids according to the invention, mention may be made of the methods mentioned above.

The present invention also relates to a nontherapeutic method for demonstrating an effect of a treatment capable of causing regression of an epithelial disorder, in particular a skin or scalp disorder, in an individual, comprising at least the steps consisting in:

a) carrying out, before the treatment, at least a first determination, in a first sample of an epithelium taken from said individual, of a biological activity and/or of the expression of a polypeptide in accordance with the invention, or of the expression of a nucleic acid sequence encoding said polypeptide,

b) carrying out, after the treatment, at least a second determination, in a second sample of an epithelium taken from said individual, of said biological activity and/or of said expression of said polypeptide or of said expression of said nucleic acid sequence, determined in step a), and

c) comparing the first and second determinations, in particular in order to deduce therefrom information relating to at least one effect of the treatment.

In particular, a method of the invention allows for demonstrating an effect of a treatment capable of causing regression of an aged, and optionally dry, state of an epithelium, in particular of an epidermis

Such a treatment may in particular be a cosmetic treatment.

In particular, the treatment of which the effect is to be evaluated may be a treatment for relieving or reducing a skin or scalp disorder linked to a dysfunction of keratinocyte proliferation and/or differentiation.

More particularly the skin disorder may be skin aging, and optionally cutanous dryness.

The biological activity and the expression of a polypeptide may be determined as indicated above.

According to another aspect, the present invention relates to a method for the cosmetic treatment of a skin disorder, comprising at least one step consisting in applying at least one cosmetic composition in accordance with the invention to at least one part of the skin, mucous membranes and/or keratin fibers.

According to another aspect, the present invention relates to the use of an effective amount of at least one polypeptide in accordance with the invention or of at least one agent for modulating the expression of said polypeptide, for the preparation of and/or for improving a pluristratified cell model, especially of epidermal or mucosal type, and in particular a reconstructed skin model.

For the purpose of the invention, the term “reconstructed skin model” is intended to denote a model in which various cell types are combined, such as in particular the natural constituents of the skin, like for example keratinocytes, fibroblasts, Langerhans cells and melanocytes.

The cells of the fibroblast type may or may not be irradiated.

Such models and the preparation thereof are known to those skilled in the art.

For the purpose of the present invention, “a” should be understood, unless otherwise indicated, in the sense of “at least one”.

The examples presented below are given by way of nonlimiting illustration of the invention.

### EXAMPLE I

### Analysis of Samples Taken by Varnish Stripping on Various Skin Regions of an Individual

The analyses are carried out using varnish strippings performed on the legs.

The individuals participating in the study are put into 4 groups.

The AS group corresponds to group 1: dry menopausal individuals, n=15.

The AN group corresponds to group 2: normal menopausal individuals, n=13.

The JS group corresponds to group 3: dry young individuals, n=16.

The JN group corresponds to group 4: normal young individuals, n=14.

1: Preparation of Acetone Powders

Two varnish strippings (B. Mehul et al., J Biol Chem 2000, Apr 28; 275(17): 12841-7) of 10 cm² are placed into 20 ml of acetone. The corneocytes become detached. The mixture is filtered through a 40 μm nylon membrane. Three successive rinses are carried out with the same volume of acetone. The suspension is finally filtered on a vacuum pump. Acetone powders of stratum corneum are obtained in dry form.

2: Sample Extraction

An extraction is carried out under denaturing conditions. To do this, a prewash is carried out with a volume (100 μl) of PBS buffer (phosphate buffered saline)+0.1% Triton X100, which is added per mg of acetone powder. The mixture is ground in a Potter and centrifuged. The corneocyte pellet is recovered. It is extracted with the same volume (100 μl/mg) of Laemmli buffer containing 0.0625 mM Tris, pH 6.8, 200 mM DTT, 2% SDS and 10% glycerol. The mixture is heated at boiling temperature for 10 minutes, and is then ground and centrifuged for 10 minutes at 10 000 g. The supernatant is recovered. A protein assay is carried out according to the Bradford technique with the Bradford reagent (Bio-Rad protein assay). The samples are adjusted to 1 mg/ml.

3: Protein Analysis by Western Blotting

The proteins are separated by SDS-PAGE electrophoresis. After semi-dry blotting onto a PVDF membrane (Immobilon-P Millipore) according to a standard protocol, the proteins are incubated with the anti-plakoglobin murine primary antibody (Progen 61005) overnight at 4° C. The second incubation is then carried out with the secondary antibody (anti-mouse IgG-HRP conjugate) (Bio-Rad), directed against the primary antibody, for 1 h30 at ambient temperature. The presence of plakoglobin on the membrane is revealed by immunodetection using the ECL Plus kit (Amersham). The membrane is then stained with amido black in order to detect the total proteins present on the membrane. The image is acquired with FluorSmax (Biorad) and the bands are quantified using the Quantity-one software (Biorad).

4: Results

The results are expressed as delta cnt*mm² of the protein of interest/delta cnt*mm² of total proteins.

### Methodology:

−2-way (age and type) analysis of variance of skin taking into account the interaction of these two factors +1-way (group) analysis of variance and Tukey's multiple comparison test. Since the normality and homoscedasticity conditions were not verified, the analysis was carried out after logarithmic transformation.

The statistical analysis was carried out with the SAS version 8.2 and SPSS version 12 software packages.

All the tests were carried out at the 5% two-sided threshold.

The table below gives the mean results and also their standard errors of the mean (sem).

| | | sem |
|---|---|---|
| group | Plakoglobin | plakoglobin |
| AS | 142 | 46 |
| AN | 77 | 21 |
| JS | 26 | 5 |
| JN | 70 | 25 |

A significant increase is noted in the expression of plakoglobin during skin aging (p=0.008).

| Listing sequences | |
|---|---|
| SEQ ID NO 1 | |
| 1 | ggcagaagcg catgaggctg tgggacttgc cttccatttg atttctgaat tgtgaaggtg |
| 61 | catgctttct ggaggcctgc acagaccttt cccacccgct ttcctgaaag aatttgacag |
| 121 | ggaaagtgag accgcaggaa ggctttttgg gggaaggttt cttcaatgct agactctctc |
| 181 | aggcagagct gcatcgggcc ccctgccttc cctgctccca atttcctgag accgcccgcc |
| 241 | cccacccaga attggggatc ttgatcaggt agccacgatg gaggtgatga acctgatgga |
| 301 | gcagcctatc aaggtgactg agtggcagca gacatacacc tacgactcgg gtatccactc |
| 361 | gggcgccaac acctgcgtgc cctccgtcag cagcaagggc atcatggagg aggatgaggc |
| 421 | ctgcgggcgc cagtacacgc tcaagaaaac caccacttac acccaggggg tgccccccag |
| 481 | ccaaggtgac ctggagtacc agatgtccac aacagccagg gccaaacggg tgcgggaggc |
| 541 | catgtgccct ggtgtgtcag gcgaggacag ctcgcttctg ctggccaccc aggtggaggg |
| 601 | gcaggccacc aacctgcagc gactggccga gccgtcccag ctgctcaagt cggccattgt |
| 661 | gcatctcatc aactaccagg atgatgccga gctggccact cgcgccctgc ccgagctcac |
| 721 | caaactgctc aacgacgagg acccggtggt ggtgaccaag gcggccatga ttgtgaacca |
| 781 | gctgtcgaag aaggaggcgt cgcggcgggc cctgatgggc tcgccccagc tggtggccgc |
| 841 | tgtcgtgcgt accatgcaga ataccagcga cctggacaca gcccgctgca ccaccagcat |
| 901 | cctgcacaac ctctcccacc accgggaggg gctgctcgcc atcttcaagt cgggtggcat |
| 961 | ccctgctctg gtccgcatgc tcagctcccc tgtggagtcg gtcctgttct atgccatcac |
| 1021 | cacgctgcac aacctgctcc tgtaccagga gggcgccaag atggccgtgc gcctggccga |
| 1081 | cgggctgcaa aagatggtgc ccctgctcaa caagaacaac cccaagttcc tggccatcac |
| 1141 | caccgactgc ctgcagctcc tggcctacgg caaccaggag agcaagctga tcatcctggc |
| 1201 | caatggtggg ccccaggccc tcgtgcagat catgcgtaac tacagttatg aaaagctgct |
| 1261 | ctggaccacc agtcgtgtgc tcaaggtgct atccgtgtgt cccagcaata agcctgccat |
| 1321 | tgtggaggct ggtgggatgc aggccctggg caagcacctg accagcaaca gcccccgcct |
| 1381 | ggtgcagaac tgcctgtgga ccctgcgcaa cctctcagat gtggccacca agcaggaggg |
| 1441 | cctggagagt gtgctgaaga ttctggtgaa tcagctgagt gtggatgacg tcaacgtcct |
| 1501 | cacctgtgcc acgggcacac tctccaacct gacatgcaac aacagcaaga acaagacgct |
| 1561 | ggtgacacag aacagcggtg tggaggctct catccatgcc atcctgcgtg ctggtgacaa |
| 1621 | ggacgacatc acggagcctg ccgtctgcgc tctgcgccac ctcactagcc gccaccctga |
| 1681 | ggccgagatg gcccagaact ctgtgcgtct caactatggc atcccagcca tcgtgaagct |
| 1741 | gctcaaccag cccaaccagt ggccactggt caaggcaacc atcggcttga tcaggaatct |
| 1801 | ggccctgtgc ccagccaacc atgccccgct gcaggaggca gcggtcatcc cccgcctcgt |
| 1861 | ccaactgctg gtgaaggccc accaggatgc ccagcgccac gtagctgcag gcacacagca |
| 1921 | gccctacacg gatggtgtga ggatggagga gattgtggag ggctgcaccg gagcactgca |
| 1981 | catcctcgcc cgggacccca tgaaccgcat ggagatcttc cggctcaaca ccattcccct |
| 2041 | gtttgtgcag ctcctgtact cgtcggtgga gaacatccag cgcgtggctg ccggggtgct |
| 2101 | gtgtgagctg gcccaggaca aggaggcggc cgacgccatt gatgcagagg gggcctcggc |
| 2161 | cccactcatg gagttgctgc actcccgcaa cgagggcact gccacctacg ctgctgccgt |
| 2221 | cctgttccgc atctccgagg acaagaaccc agactaccgg aagcgcgtgt ccgtggagct |
| 2281 | caccaactcc ctcttcaagc atgacccggc tgcctgggag gctgcccaga gcatgattcc |
| 2341 | catcaatgag ccctatggag atgacttgga tgccacctac cgccccatgt actccagcga |
| 2401 | tgtgcccctt gacccgctgg agatgcacat ggacatggat ggagactacc ccatcgacac |
| 2461 | ctacagcgac ggcctcaggc ccccgtaccc cactgcagac cacatgctgg cctaggcggc |
| 2521 | ctggccccag tgcggttcct catctgagag gctctccgtg caggcgatgg ggcaagacag |
| 2581 | aaaagtgcct gagctgggga agccggggtg taacttcctg ctgcaccctg cgcctccaga |
| 2641 | ggtcctccgt agggtctttc ttgggatagt gttctgctcc tgcttttctg tcctgggcat |
| 2701 | gggtccaggg cctgacaccc cctccagggc ccaggttcga gacccaaacc cccaaaatcc |
| 2761 | aaaacttctc ttgaaaagtt cagggaccgt ccaggggaga tggggaggag atatggagtg |
| 2821 | agtcacctgc tccagaagat gccagcttct ctctccaggg tgcttagttg gctttgccca |
| 2881 | cccctcactc cccagggagc tctggggaca gcttcctcac acccctgtcc cacccacaca |
| 2941 | gctgccctag ctgaccccga gaagtgctct tggctgaccc ctctggtgtg tggtgagggg |
| 3001 | ctttctcttc cccttcctgt ttcagacccc cccatttccc gcacatggtg tggggggctg |
| 3061 | ggggaggtcc aagcagagtg ttttattatt atcgctttat gtttttggtt attggttttt |
| 3121 | ttgtatagac caaagcaaag aaaataaaaa taacaaaaaa aaaaaaaaaa aaaaaaaa |
| SEQ ID NO 2 | |
| 1 | MEVMNLMEQP IKVTEWQQTY TYDSGIHSGA NTCVPSVSSK GIMEEDEACG RQYTLKKTTT |
| 61 | YTQGVPPSQG DLEYQMSTTA RAKRVREAMC PGVSGEDSSL LLATQVEGQA TNLQRLAEPS |
| 121 | QLLKSAIVHL INYQDDAELA TRALPELTKL LNDEDPVVVT KAAMIVNQLS KKEASRRALM |
| 181 | GSPQLVAAVV RTMQNTSDLD TARCTTSILH NLSHHREGLL AIFKSGGIPA LVRMLSSPVE |
| 241 | SVLFYAITTL HNLLLYQEGA KMAVRLADGL QKMVPLLNKN NPKFLAITTD CLQLLAYGNQ |
| 301 | ESKLIILANG GPQALVQIMR NYSYEKLLWT TSRVLKVLSV CPSNKPAIVE AGGMQALGKH |
| 361 | LTSNSPRLVQ NCLWTLRNLS DVATKQEGLE SVLKILVNQL SVDDVNVLTC ATGTLSNLTC |
| 421 | NNSKNKTLVT QNSGVEALIH AILRAGDKDD ITEPAVCALR HLTSRHPEAE MAQNSVRLNY |
| 481 | GIPAIVKLLN QPNQWPLVKA TIGLIRNLAL CPANHAPLQE AAVIPRLVQL LVKAHQDAQR |
| 541 | HVAAGTQQPY TDGVRMEEIV EGCTGALHIL ARDPMNRMEI FRLNTIPLFV QLLYSSVENI |
| 601 | QRVAAGVLCE LAQDKEAADA IDAEGASAPL MELLHSRNEG TATYAAAVLF RISEDKNPDY |
| 661 | RKRVSVELTN SLFKHDPAAW EAAQSMIPIN EPYGDDLDAT YRPMYSSDVP LDPLEMHMDM |
| 721 | DGDYPIDTYS DGLRPPYPTA DHMLA |
| SEQ ID NO 3 | |
| | NSGVEALIHAILR |
| SEQ ID NO 4 | |
| | TQNSGVEALIHAILR |
| SEQ ID NO 5 | |
| | VTQNSGVEALIHAILR |

1. A cosmetic method for preventing and/or treating aged skins, comprising:
applying to the skin of an individual in need thereof, an effective amount of a material selected from the group consisting of at least one polypeptide having an amino acid sequence encoded by a nucleic acid sequence represented entirely or partly by a sequence represented by SEQ ID NO 1, an analog thereof or a fragment thereof, at least one nucleic sequence encoding the at least one polypeptide or of at least one agent for modulating the activity, the stability or the expression of the at least one polypeptide.

2. A therapeutic method for preventing an/or treating aged skins, comprising:
application to an individual in need thereof of a therapeutic composition prepared with an effective amount of a material selected from the group consisting of at least one polypeptide having an amino acid sequence encoded by a nucleic acid sequence represented entirely or partly by a sequence represented by SEQ ID NO 1, an analog thereof or a fragment thereof, of at least one nucleic sequence encoding the at least one polypeptide or of at least one agent for modulating the activity, the stability or the expression of the at least one polypeptide.

3. The method as claimed in claim 1, in which said polypeptide has an amino acid sequence represented entirely or partly by a sequence represented by SEQ ID NO 2, an analog thereof or a fragment thereof.

4. The method as claimed in claim 3, in which said polypeptide has an amino acid sequence chosen from SEQ ID NO 3, SEQ ID NO 4 and SEQ ID NO 5.

5. The method as claimed in claim 1, wherein the modulating agent is an inhibitor of the gene expression of said polypeptide.

6. The method as claimed in the preceding claim 5, wherein said inhibiting agent is selected from the group consisting of HMGN1, HOXD3, Brn-3b, TNF-alpha, TCDD, alpha-3, beta-1 integrin, cadherin 11 and ALK-1.

7. The method as claimed in claim 1, wherein the modulating agent is an agent for decreasing the stability of said polypeptide.

8. The method as claimed in claim 1, wherein said composition is for preventing and/or treating thinning of an epidermis and/or a loss of firmness, of elasticity, of density and/or of tonicity of an epidermis and/or the formation of wrinkles and fine lines.

9. The method as claimed in claim 1, wherein said composition is for preventing and/or treating cutaneous signs of dryness.

10. The method as claimed in claim 1, wherein said composition is for preventing and/or treating disorders of the barrier function of an epidermis.

11. A method for screening for biological or chemical compounds capable of modulating the expression and/or the biological activity of at least one polypeptide as defined according to claim 1, comprising contacting the polypeptide with said biological or chemical compound.

12. The method for characterizing, in vitro or ex vivo, an aged state of an epithelium comprising determining in a sample, a content of at least one polypeptide as defined according to claim 1, or of at least one nucleic acid sequence encoding said polypeptide.

13. The method as claimed claim 12, wherein the state of the epithelium is associated with dryness.

14. A method for characterizing an aged state of an epithelium, comprising:
a) determining, in a sample of said epithelium, the content of a polypeptide as defined according to claim 1 or of a nucleic acid sequence encoding said polypeptide, and
b) comparing said content determined in step a) to a reference value.

15. The method as claimed in claim 14, wherein the method is noninvasive.

16. A method for screening for anti-aging active agents, comprising:
a) bringing at least one cell type capable of expressing a polypeptide as defined according to claim 1 into contact with at least one chemical or biological test compound, under conditions suitable for manifestation of the expression of said polypeptide,
b) determining the content of said polypeptide.

17. A cosmetic method for characterizing the effectiveness of a cosmetic or therapeutic treatment aiming to compensate for the signs of skin aging, comprising the qualitative or quantitative characterization of the expression and/or of the biological activity of a polypeptide according to claim 1.

18. A method for the preparation of and/or improving a pluristratified cell model comprising contacting said pluristratified cell model with an effective amount of at least one polypeptide according to claim 1, or of at least one agent for modulating the expression of said polypeptide.

19. The method as claimed in claim 2, wherein said polypeptide has an amino acid sequence represented entirely or partly by a sequence represented by SEQ ID NO 2, an analog thereof or a fragment thereof.

20. The method as claimed in claim 19, wherein said polypeptide has an amino acid sequence chosen from SEQ ID NO 3, SEQ ID NO 4 and SEQ ID NO 5.

21. The method as claimed in claim 2, wherein the modulating agent is an inhibitor of the gene expression of said polypeptide.

22. The method as claimed in claim 21, wherein said inhibiting agent is selected from the group consisting of HMGN1, HOXD3, Brn-3b, TNF-alpha, TCDD, alpha-3, beta-1 integrin, cadherin 11 and ALK-1.

23. The method as claimed in claim 2, wherein the modulating agent is an agent for decreasing the stability of said polypeptide.

24. The method as claimed in claim 2, wherein said composition is for preventing and/or treating thinning of an epidermis and/or a loss of firmness, of elasticity, of density and/or of tonicity of an epidermis and/or the formation of wrinkles and fine lines.

25. The method as claimed in claim 2, wherein said composition is for preventing and/or treating cutaneous signs of dryness.

26. The method as claimed in claim 2, wherein said composition is for preventing and/or treating disorders of the barrier function of an epidermis.


SEQUENCE LISTING (5 sequences)

SEQ ID NO: 1 - DNA, 3178 bp, Homo sapiens
ggcagaagcgcatgaggctgtgggacttgccttccatttgatttctgaattgtgaaggtgcatgctttctggaggcctgcacagacctttcccacccgctttcctgaaagaatttgacagggaaagtgagaccgcaggaaggctttttgggggaaggtttcttcaatgctagactctctcaggcagagctgcatcgggccccctgccttccctgctcccaatttcctgagaccgcccgcccccacccagaattggggatcttgatcaggtagccacgatggaggtgatgaacctgatggagcagcctatcaaggtgactgagtggcagcagacatacacctacgactcgggtatccactcgggcgccaacacctgcgtgccctccgtcagcagcaagggcatcatggaggaggatgaggcctgcgggcgccagtacacgctcaagaaaaccaccacttacacccagggggtgccccccagccaaggtgacctggagtaccagatgtccacaacagccagggccaaacgggtgcgggaggccatgtgccctggtgtgtcaggcgaggacagctcgcttctgctggccacccaggtggaggggcaggccaccaacctgcagcgactggccgagccgtcccagctgctcaagtcggccattgtgcatctcatcaactaccaggatgatgccgagctggccactcgcgccctgcccgagctcaccaaactgctcaacgacgaggacccggtggtggtgaccaaggcggccatgattgtgaaccagctgtcgaagaaggaggcgtcgcggcgggccctgatgggctcgccccagctggtggccgctgtcgtgcgtaccatgcagaataccagcgacctggacacagcccgctgcaccaccagcatcctgcacaacctctcccaccaccgggaggggctgctcgccatcttcaagtcgggtggcatccctgctctggtccgcatgctcagctcccctgtggagtcggtcctgttctatgccatcaccacgctgcacaacctgctcctgtaccaggagggcgccaagatggccgtgcgcctggccgacgggctgcaaaagatggtgcccctgctcaacaagaacaaccccaagttcctggccatcaccaccgactgcctgcagctcctggcctacggcaaccaggagagcaagctgatcatcctggccaatggtgggccccaggccctcgtgcagatcatgcgtaactacagttatgaaaagctgctctggaccaccagtcgtgtgctcaaggtgctatccgtgtgtcccagcaataagcctgccattgtggaggctggtgggatgcaggccctgggcaagcacctgaccagcaacagcccccgcctggtgcagaactgcctgtggaccctgcgcaacctctcagatgtggccaccaagcaggagggcctggagagtgtgctgaagattctggtgaatcagctgagtgtggatgacgtcaacgtcctcacctgtgccacgggcacactctccaacctgacatgcaacaacagcaagaacaagacgctggtgacacagaacagcggtgtggaggctctcatccatgccatcctgcgtgctggtgacaaggacgacatcacggagcctgccgtctgcgctctgcgccacctcactagccgccaccctgaggccgagatggcccagaactctgtgcgtctcaactatggcatcccagccatcgtgaagctgctcaaccagcccaaccagtggccactggtcaaggcaaccatcggcttgatcaggaatctggccctgtgcccagccaaccatgccccgctgcaggaggcagcggtcatcccccgcctcgtccaactgctggtgaaggcccaccaggatgcccagcgccacgtagctgcaggcacacagcagccctacacggatggtgtgaggatggaggagattgtggagggctgcaccggagcactgcacatcctcgcccgggaccccatgaaccgcatggagatcttccggctcaacaccattcccctgtttgtgcagctcctgtactcgtcggtggagaacatccagcgcgtggctgccggggtgctgtgtgagctggcccaggacaaggaggcggccgacgccattgatgcagagggggcctcggccccactcatggagttgctgcactcccgcaacgagggcactgccacctacgctgctgccgtcctgttccgcatctccgaggacaagaacccagactaccggaagcgcgtgtccgtggagctcaccaactccctcttcaagcatgacccggctgcctgggaggctgcccagagcatgattcccatcaatgagccctatggagatgacttggatgccacctaccgccccatgtactccagcgatgtgccccttgacccgctggagatgcacatggacatggatggagactaccccatcgacacctacagcgacggcctcaggcccccgtaccccactgcagaccacatgctggcctaggcggcctggccccagtgcggttcctcatctgagaggctctccgtgcaggcgatggggcaagacagaaaagtgcctgagctggggaagccggggtgtaacttcctgctgcaccctgcgcctccagaggtcctccgtagggtctttcttgggatagtgttctgctcctgcttttctgtcctgggcatgggtccagggcctgacaccccctccagggcccaggttcgagacccaaacccccaaaatccaaaacttctcttgaaaagttcagggaccgtccaggggagatggggaggagatatggagtgagtcacctgctccagaagatgccagcttctctctccagggtgcttagttggctttgcccacccctcactccccagggagctctggggacagcttcctcacacccctgtcccacccacacagctgccctagctgaccccgagaagtgctcttggctgacccctctggtgtgtggtgaggggctttctcttccccttcctgtttcagacccccccatttcccgcacatggtgtggggggctgggggaggtccaagcagagtgttttattattatcgctttatgtttttggttattggtttttttgtatagaccaaagcaaagaaaataaaaataacaaaaaaaaaaaaaaaaaaaaaaaa

SEQ ID NO: 2 - protein, 745 aa, Homo sapiens
MEVMNLMEQPIKVTEWQQTYTYDSGIHSGANTCVPSVSSKGIMEEDEACGRQYTLKKTTTYTQGVPPSQGDLEYQMSTTARAKRVREAMCPGVSGEDSSLLLATQVEGQATNLQRLAEPSQLLKSAIVHLINYQDDAELATRALPELTKLLNDEDPVVVTKAAMIVNQLSKKEASRRALMGSPQLVAAVVRTMQNTSDLDTARCTTSILHNLSHHREGLLAIFKSGGIPALVRMLSSPVESVLFYAITTLHNLLLYQEGAKMAVRLADGLQKMVPLLNKNNPKFLAITTDCLQLLAYGNQESKLIILANGGPQALVQIMRNYSYEKLLWTTSRVLKVLSVCPSNKPAIVEAGGMQALGKHLTSNSPRLVQNCLWTLRNLSDVATKQEGLESVLKILVNQLSVDDVNVLTCATGTLSNLTCNNSKNKTLVTQNSGVEALIHAILRAGDKDDITEPAVCALRHLTSRHPEAEMAQNSVRLNYGIPAIVKLLNQPNQWPLVKATIGLIRNLALCPANHAPLQEAAVIPRLVQLLVKAHQDAQRHVAAGTQQPYTDGVRMEEIVEGCTGALHILARDPMNRMEIFRLNTIPLFVQLLYSSVENIQRVAAGVLCELAQDKEAADAIDAEGASAPLMELLHSRNEGTATYAAAVLFRISEDKNPDYRKRVSVELTNSLFKHDPAAWEAAQSMIPINEPYGDDLDATYRPMYSSDVPLDPLEMHMDMDGDYPIDTYSDGLRPPYPTADHMLA

SEQ ID NO: 3 - protein, 13 aa, Artificial Sequence
  Description of Artificial Sequence Synthetic peptide
NSGVEALIHAILR

SEQ ID NO: 4 - protein, 15 aa, Artificial Sequence
  Description of Artificial Sequence Synthetic peptide
TQNSGVEALIHAILR

SEQ ID NO: 5 - protein, 16 aa, Artificial Sequence
  Description of Artificial Sequence Synthetic peptide
VTQNSGVEALIHAILR